(19) United States
(12) Patent Application Publication
(10) Pub. No.: US 2023/0087452 A1
(43) Pub. Date: Mar. 23, 2023

(54) DEVICES AND METHODS FOR ATTACHING NON-CONNECTED ANATOMICAL STRUCTURES

(71) Applicant: BOSTON SCIENTIFIC SCIMED, INC., Maple Grove, MN (US)

(72) Inventors: Gary Gilmartin, Foxford (IE); Martyn G. Folan, Galway (IE)

(73) Assignee: BOSTON SCIENTIFIC SCIMED, INC., Maple Grove, MN (US)

(21) Appl. No.: 17/941,867

(22) Filed: Sep. 9, 2022

Related U.S. Application Data
(60) Provisional application No. 63/246,376, filed on Sep. 21, 2021.

(51) Int. Cl. A61F 2/04; A61F 5/00

(52) U.S. Cl. CPC A61F 2/04; A61F 5/0076; A61B 17/1114

(57) ABSTRACT

An illustrative stent may include a tubular member defined by at least one knitted filament forming a plurality of twisted knit stitches with rungs extending circumferentially between radially adjacent twisted knit stitches, where each twisted knit stitch is interconnected with a longitudinally adjacent twisted knit stitch forming a series of linked stitches. The tubular member may be implanted to connect two spaced apart anatomical locations within the digestive tract, moving between a relaxed configuration and an elongated configuration. The tubular member has a first longitudinal length in the relaxed configuration and a second longitudinal length in the elongated configuration, and the first longitudinal length is less than the second longitudinal length.

### CROSS-REFERENCE TO RELATED APPLICATIONS

This application claims the benefit of U.S. Provisional Pat. Application Serial No. 63/246,376, filed Sep. 21, 2021, which is incorporated herein by reference.

### TECHNICAL FIELD

The present disclosure pertains to medical devices, methods for manufacturing medical devices, and uses thereof. More particularly, the present disclosure pertains to a stent for implantation in body lumens, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc.

In some instances it may be desirable to design stents to include sufficient flexibility while maintaining sufficient radial force to open the body lumen at the treatment site. However, in some stents, the compressible and flexible properties that assist in stent delivery may also result in a stent that has a tendency to migrate from its originally deployed position. For example, stents that are designed to be positioned in the gastrointestinal and biliary tract may have a tendency to migrate due to peristalsis (i.e., the involuntary constriction and relaxation of the muscles of the stomach, intestine, and colon). Additionally, the generally moist and inherently lubricious environment of the stomach, intestine, colon, etc. further contributes to a stent’s tendency to migrate when deployed therein.

Various medical procedures involve the temporary or permanent joining of non-connected anatomical structures. Some examples include a hepaticogastrostomy (HGS) involving joining the hepatic duct and the stomach to drain the bile duct, a gastrojejeneum (GJ) bypass procedure to create an anastomosis between the small intestine and stomach wall, and stomas to create an artificial opening into the large intestine or other region of the digestive tract. In these medical procedures, peristalsis and gross organ movement in one or both of the anatomical structures being connected may create difficulties in using stents to join the structures due to stent migration.

Therefore, it may be desirable to design a stent with anti-migration features to reduce the stent’s tendency to migrate. Examples of medical devices including anti-migration features, and methods of using them are disclosed herein.

### SUMMARY

This disclosure provides design, material, manufacturing method, and use alternatives for medical devices. An example stent configured to connect two spaced apart anatomical locations comprises an elongated tubular member having a longitudinal axis, the elongated tubular member comprising at least one knitted filament forming a plurality of twisted knit stitches with rungs extending circumferentially between radially adjacent twisted knit stitches, wherein each twisted knit stitch is interconnected with a longitudinally adjacent twisted knit stitch forming a series of linked stitches, the elongated tubular member configured to move between a relaxed configuration and an elongated configuration, wherein each of the plurality of twisted knit stitches is formed by a single filament defining a loop portion and a crossed base region, and wherein the elongated tubular member has a first longitudinal length in the relaxed configuration and a second longitudinal length in the elongated configuration, wherein the first longitudinal length is less than the second longitudinal length.

Alternatively or additionally to the embodiment above, the elongated tubular member has a first outer diameter in the relaxed configuration and a second outer diameter in the elongated configuration, wherein the first and second outer diameters are substantially the same.

Alternatively or additionally to any of the embodiments above, the first and second outer diameters are defined by the rungs.

Alternatively or additionally to any of the embodiments above, the loop portion of at least some of the twisted knit stitches is wrapped around the crossed base region of the longitudinally adjacent twisted knit stitch.

Alternatively or additionally to any of the embodiments above, when in the relaxed configuration the rungs define an outer surface of the elongated tubular member and the crossed base region of each twisted knit stitch extends radially outward from the outer surface.

Alternatively or additionally to any of the embodiments above, the crossed base regions form a raised ridge extending helically around the elongated tubular member in the relaxed configuration.

Alternatively or additionally to any of the embodiments above, the at least one knitted filament is only a single knitted filament.

An example method of providing an artificial bridge between a first organ and a second organ of a patient comprises securing a first end of an expandable stent to the first organ and securing a second end of the stent to the second organ such that an interior of the first organ is in fluid communication with an interior of the second organ through the stent, wherein the first and second organs are spaced apart and at least one of the first and second organs moves relative to the other of the first and second organ during normal function of the first and second organs, wherein the stent comprises an elongated tubular member having a longitudinal axis, the elongated tubular member comprising at least one knitted filament forming a plurality of twisted knit stitches with rungs extending circumferentially between circumferentially adjacent twisted knit stitches, wherein each twisted knit stitch is interconnected with a longitudinally adjacent twisted knit stitch forming a series of linked stitches, the elongated tubular member configured to move between a relaxed configuration and an elongated configuration, wherein each of the plurality of twisted knit stitches is formed by a single filament defining a loop portion and a crossed base region, and wherein the elongated tubular member has a first longitudinal length in the relaxed configuration and a second longitudinal length in the elongated configuration, wherein the first longitudinal length is less than the second longitudinal length.

Alternatively or additionally to the embodiment above, the first organ is a hepatic duct and the second organ is the patient’s stomach.

Alternatively or additionally to any of the embodiments above, the stent incudes an uncoated portion and a coated portion, wherein the coated portion includes at least 70% of a length of the stent and the coated portion includes a fluid impervious coating, wherein the uncoated portion extends into the hepatic duct.

Alternatively or additionally to any of the embodiments above, the first organ is the patient’s stomach and the second organ is a portion of the patient’s small intestine.

Alternatively or additionally to any of the embodiments above, the first organ is the patient’s skin and the second organ is a portion of the patient’s large intestine.

Alternatively or additionally to any of the embodiments above, the first end of the stent includes a flange disposed on an outer surface of the skin.

Alternatively or additionally to any of the embodiments above, the second end of the stent is disposed coaxially within the large intestine.

Alternatively or additionally to any of the embodiments above, the second end of the stent disposed coaxially within the large intestine includes an outwardly flared region.

Alternatively or additionally to any of the embodiments above, the patient’s stomach includes a stomach pouch portion separated from an excluded stomach portion, and a Roux limb connecting the stomach pouch portion with the patient’s small intestine, wherein the first organ is the stomach pouch portion and the second organ is the excluded stomach portion.

Alternatively or additionally to any of the embodiments above, after the stent is secured, the method further comprises inserting an endoscope into the stomach pouch portion, through the stent into the excluded stomach portion, and into the patient’s duodenum, followed by performing an endoscopic retrograde cholangiopancreatography (ERCP).

Alternatively or additionally to any of the embodiments above, after ERCP is completed, the method further comprises removing the stent.

Another example method of draining fluids from a first organ to a second organ within a patient’s digestive tract comprises inserting an implantable stent within the patient, wherein the implantable stent includes a tubular member formed from at least one knitted wire filament, forming a plurality of twisted knit stitches with rungs extending circumferentially between circumferentially adjacent twisted knit stitches, wherein each twisted knit stitch includes a loop portion and a crossed base region and is interconnected with a longitudinally adjacent twisted knit stitch forming a series of linked stitches, the tubular member configured to move between a relaxed configuration with a first longitudinal length and a first diameter, and an elongated configuration with a second longitudinal length and a second diameter, wherein the first longitudinal length is less than the second longitudinal length and the first and second diameters are substantially the same, and positioning a first end of the tubular member within the first organ of the patient and a second end of the tubular member within the second organ of the patient, wherein the first and second organs are spaced apart from one another and move relative one another during digestion.

Alternatively or additionally to the embodiment above, when in the relaxed configuration the rungs define an outer surface of the tubular member and the crossed base region of each twisted knit stitch extends radially outward from the outer surface.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 illustrates part of the digestive tract;

FIGS. 2A and 2B illustrate movement of the stomach relative to the hepatic duct;

FIGS. 3A and 3B illustrate prior art braided stents;

FIG. 4 illustrates a portion of a prior art parallel knitted stent pattern;

FIG. 5 is a perspective view of an illustrative stent in a relaxed configuration;

FIG. 6 is an enlarged top view of a portion of the illustrative stent of FIG. 5;

FIG. 7 is an illustration of the stent of FIG. 5 in an elongated configuration in a delivery sheath;

FIG. 8 is an enlarged view of a portion of the stent of FIG. 7;

FIG. 9 is an enlarged side view of a longitudinal edge of the illustrative stent of FIG. 5;

FIG. 10 is an illustration of a portion of the stent of FIG. 5 disposed within a body organ;

FIG. 11A is an illustration of a prior art braided stent in a relaxed configuration;

FIG. 11B is an illustration of the stent of FIG. 11A in an elongated configuration;

FIG. 12A is an illustration of the stent of FIG. 5 in a relaxed configuration;

FIG. 12B is an illustration of the stent of FIG. 12A in an elongated configuration;

FIG. 13 is an illustration of another illustrative stent with a flared end;

FIG. 14 illustrates an example stent connecting the hepatic duct and stomach;

FIG. 15 illustrates an example stent connecting the stomach and small intestine;

FIG. 16 illustrates an example stent connecting the large intestine to the skin surface; and

FIG. 17 illustrates an example stent connecting a stomach pouch to the excluded stomach portion with an endoscope extending through the stent during a subsequent endoscopic retrograde cholangiopancreatography (ERCP) procedure.

While the disclosure is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

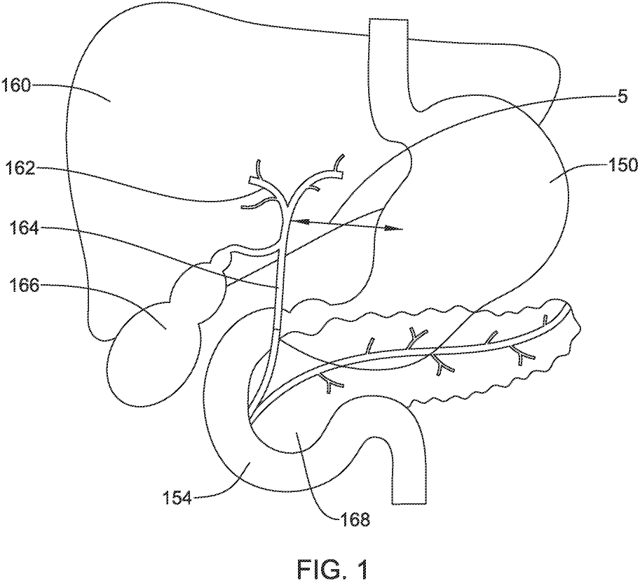

FIG. 1 illustrates various organs in the digestive tract, including the stomach 150, duodenum 154, liver 160, hepatic duct 162, gallbladder 166, common bile duct 164, and pancreas 168.

Bile, which is produced in the liver 160, flows through a series of hepatic ducts 162 that drain into one large duct called the common bile duct (CBD) 164. The CBD then connects to the duodenum 154, allowing the bile to flow into the duodenum for digestion. If the hepatic or bile ducts becomes blocked, bile cannot drain normally and backs up or builds up in the liver. Blocked bile ducts may cause jaundice, dark urine, nausea and poor appetite, leading to potentially serious conditions.

Endoscopic retrograde cholangiopancreatography (ERCP) may be used to diagnose and treat conditions of the bile ducts, including, for example, gallstones, inflammatory strictures, leaks (e.g., from trauma, surgery, etc.), and cancer. Blockage of the biliary duct may occur in many of the disorders of the biliary system, including the disorders of the liver, such as, primary schlerosing cholangitis, stone formation, scarring in the duct, etc. Draining blocked fluids from the biliary system may be used to treat the disorders. Methods of biliary drainage include the placement of plastic or metal stents to relieve the blockage. In the case of a gallstone causing the obstruction in the duct, a number of products are also available to resolve this through ERCP. However, access to the bile ducts via ERCP may not be possible due to a variety of reasons such as a tumor blocking the passageway, anatomic variation, periampullary diverticula, etc.

When ERCP methods prove unsuccessful, percutaneous drainage (PTCD) can be performed. However, PTCD may be associated with complications such as bleeding and bile leakage. If subsequent internal drainage cannot be achieved, the patient would have to accept long-term external biliary drainage, which can be uncomfortable and have significant impairment of quality of life.

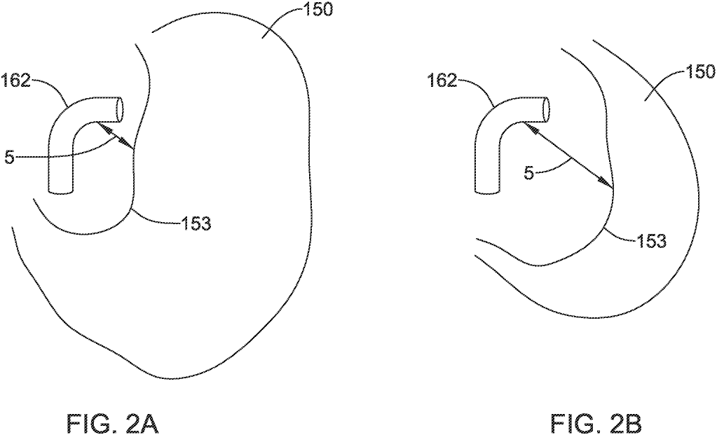

Endoscopic Ultrasound (EUS) guided biliary drainage (BD) offers an alternative option to surgery and percutaneous drainage for treating obstructive jaundice when ERCP drainage fails. Hepaticogastrostomy (HGS) may be performed to join the hepatic duct 162 to the stomach 150. This would allow the build-up of bile to flow into the stomach and may relieve the symptoms caused by bile buildup, i.e. jaundice. However, the hepatic duct 162 and the stomach 150 are spaced apart by a distance 5 indicated by the arrow in FIG. 1. The distance 5 between the organs requires a relatively long stent. Additionally, as the stomach muscles contract to churn food, the distance 5 between the gastric wall 153 of the stomach 150 relative to the hepatic duct 162 varies, from a relatively small distance 5 when the stomach is relaxed as shown in FIG. 2Ato a greater distance 5 when the stomach is contracted as shown in FIG. 2B. In addition to the gastric wall flexing, the stomach undergoes peristalsis during digestion. This relative motility of the stomach is understood to be complex, in three dimensions rather than in an exclusively linear manner. The distance between target organs to be joined, as well as the relative movement of at least one of the organs, may increase the chance of stent migration.

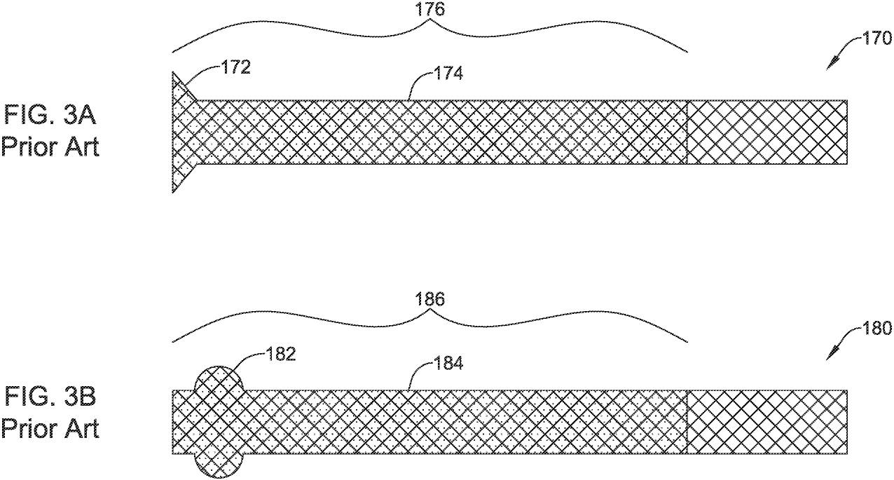

Prior art braided stents 170 and 180 such as those illustrated in FIGS. 3A and 3B may be used in HGS. The braided stents 170, 180 may be self-expanding metal stents with a proximal flange 172, 182 and may have a coating 174, 184 over, typically, 70% of the length of the stent. The coated portion or length 176, 186 may be configured to span the peritoneal space between the stomach and hepatic duct, preventing bile leakage into the peritoneum and creating a sealed channel between the two organs. The 30% uncoated distal portion of the stent 170, 180 may be configured to allow initial, acute side branch flow of bile into the stent, and in a post-acute situation, allow tissue in-growth into the stent to reduce migration. However, migration can still occur, possibly due to the movement of the stomach relative to the hepatic duct.

For HGS patients, stent migration may cause serious complications including death. If there is no tissue ingrowth-based adhesion at the hepatic duct, the stent may migrate proximally into the stomach, causing leakage of biliary contents into the peritoneum, resulting in peritonitis. If there is sufficient or excessive adhesion at the hepatic duct, the stent may migrate distally into the peritoneum, causing leakage of biliary and stomach contents into the peritoneum, also causing peritonitis. Additionally, the now migrated stent is free to abrade the outer gastric wall and other organs or vessels in the vicinity.

Anatomically, stent migration can occur as a result of the hepatic duct 162 being a generally static vessel, whereas the stomach 150 is a highly motile vessel, as illustrated in FIG. 2. This movement can cause the stent to migrate as the gastric wall slides/pulls on the proximal flange of the stent during stomach contraction. Braided stents reduce in diameter (constrain) when longitudinal force is applied, which can exacerbate migration. This phenomenon is particularly evident during stent removal procedures. Additionally, some knitted stent designs may be similarly problematic as while much higher forces are required to cause the knit to reduce in diameter (constrain) this results in a fixed device length prone to migration and delivered by a less conducive crocheted delivery system.

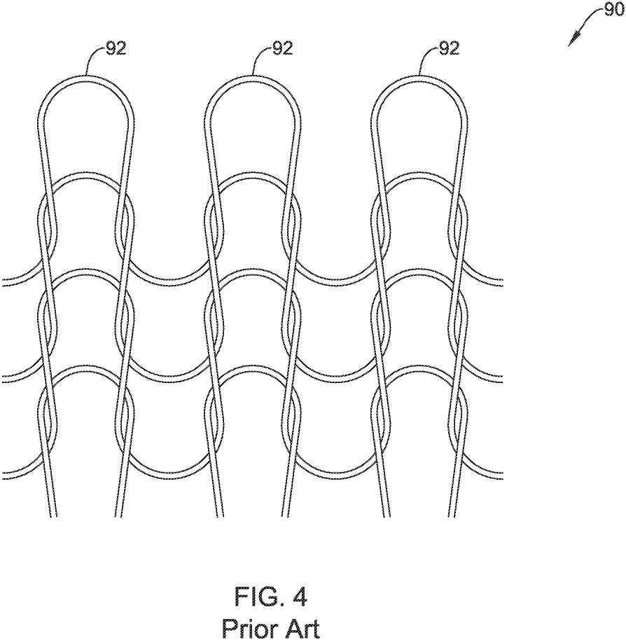

FIG. 4 illustrates a portion of a prior art self-expanding knitted stent 90. Conventional knitted self-expanding stents are generally designed using an automated weft knitting process that produces parallel columns 92 of knit stitches that run parallel to the longitudinal axis of the stent in both the expanded, relaxed configuration and the elongated, constrained configuration. The parallel knitted stent 90 provides good radial strength with minimal foreshortening. However, this parallel knitted stent design may be difficult to constrain, especially into a coaxial delivery system and thus may be delivered using a system which may not offer a method of recapture, such as a crochet delivery system. Additionally, parallel knitted stent designs have a tendency to migrate in-situ. An alternative knitted self-expanding stent is desired that has similar conformability and radial forces as previous parallel knitted stent configurations, but resists migration and can adapt its length without significant lumen diameter reduction to leave an intact channel for drainage.

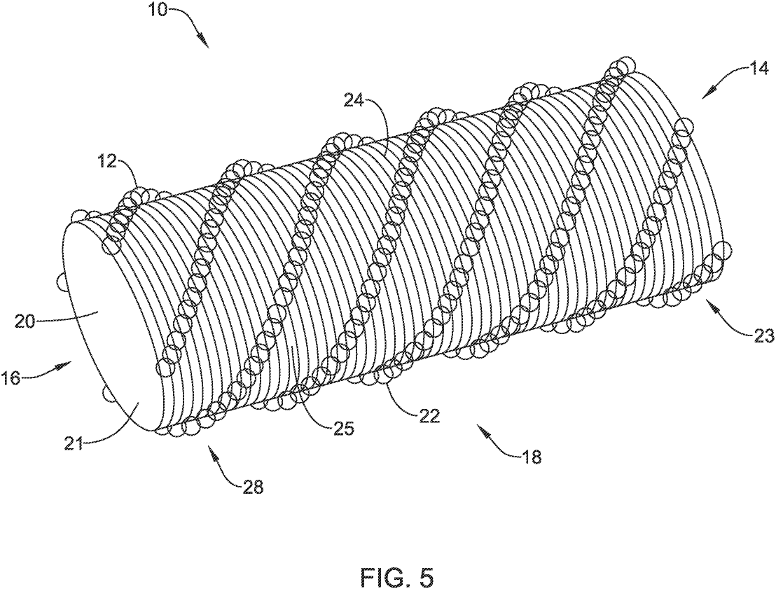

FIG. 5 illustrates a perspective view of an example implant for creating an artificial bridge to connect adjacent organs, such as, but not limited to, a stent 10. In some instances, the stent 10 may be an expandable stent formed as an elongated tubular member 12 with a helical design. While the stent 10 is described as generally tubular, it is contemplated that the stent 10 may take any cross-sectional shape desired. The stent 10 may have a first, or proximal end 14, a second, or distal end 16, and an intermediate region 18 disposed between the proximal end 14 and the distal end 16. The stent 10 may include a lumen 20 extending from a first opening adjacent the proximal end 14 to a second opening adjacent to the distal end 16 to allow for the passage of fluids, etc.

The stent 10 may be fabricated from at least one filament 24 defining open cells 25 and twisted knit stitches 22. In some examples, the stent 10 may be formed from only a single filament 24 interwoven with itself to form open cells 25 and twisted knit stitches 22. In some cases, the filament 24 may be a monofilament, while in other cases the filament 24 may be two or more filaments wound, braided, or woven together. In some instances, an inner surface of the stent 10 may be entirely, substantially or partially, covered with a fluid impervious covering or coating 21. In other embodiments, the coating 21 may be disposed on the outer surface of the stent. In some embodiments, the covering may be a polymer. The covering or coating may extend across and/or occlude one or more, or a plurality of the open cells 25 and twisted knit stitches 22 defined by the filament 24. The covering or coating may prevent bile leakage into the peritoneum.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, Nitinol and Elgiloy®. Depending on the material selected for construction, the stent 10 may be self-expanding (i.e., configured to automatically radially expand when unconstrained). In some embodiments, fibers may be used to make the stent 10, which may be composite fibers, for example, having an outer shell made of Nitinol having a platinum core. It is further contemplated the stent 10 may be formed from polymers including, but not limited to, polyethylene terephthalate (PET). The stent 10 may be self-expanding. As used herein the term “self-expanding” refers to the tendency of the stent to return to a preprogrammed diameter when unrestrained from an external biasing force (for example, but not limited to a delivery catheter or sheath). In some instances, in the relaxed, expanded configuration as shown in FIG. 5, the stent 10 may include a first end region 23 proximate the proximal end 14 and a second end region 28 proximate the distal end 16.

In some embodiments, the stent 10 may have a uniform outer diameter from the proximal end 14 to the distal end 16 when in the relaxed, expanded configuration, as shown in FIG. 5. In some embodiments, the outer diameter of the intermediate region 18 may be in the range of 15 to 25 millimeters. The outer diameter of the anti-migration flares (proximal end 14 and/or distal end 16) may be in the range of 20 to 30 millimeters. It is contemplated that the outer diameter of the stent 10 may be varied to suit the desired application.

It is contemplated that the stent 10 can be made from a number of different materials such as, but not limited to, metals, metal alloys, shape memory alloys and/or polymers, as desired, enabling the stent 10 to be expanded into shape when accurately positioned within the body. In some instances, the material may be selected to enable the stent 10 to be removed with relative ease as well. For example, the stent 10 can be formed from alloys such as, but not limited to, Nitinol and Elgiloy®. Depending on the material selected for construction, the stent 10 may be self-expanding or require an external force to expand the stent 10. In some embodiments, composite filaments may be used to make the stent 10, which may include, for example, an outer shell or cladding made of Nitinol and a core formed of platinum or other radiopaque material. It is further contemplated the stent 10 may be formed from polymers including, but not limited to, polyethylene terephthalate (PET). In some instances, the filaments of the stent 10, or portions thereof, may be bioabsorbable or biodegradable, while in other instances the filaments of the stent 10, or portions thereof, may be biostable.

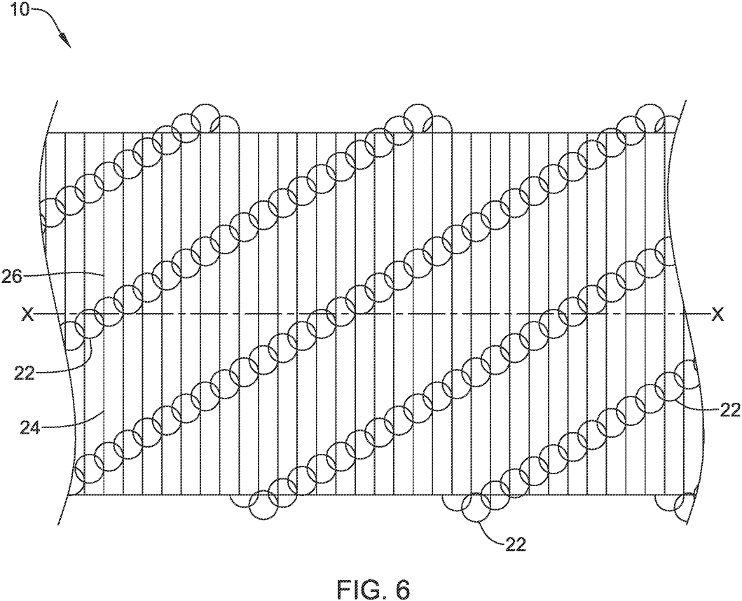

FIG. 6 illustrates details of the helical structure of the stent 10 when in the relaxed, expanded configuration. The stent 10 as illustrated may be fabricated from a single filament 24 forming twisted knit stitches 22 separated by elongate rungs 26 extending circumferentially between circumferentially adjacent twisted knit stitches 22. Each twisted knit stitch 22 may be interconnected with a longitudinally adjacent twisted knit stitch 22 forming a series of linked stitches that extend helically around the stent in the relaxed, expanded configuration, as shown in FIG. 6. The interconnected twisted knit stitches 22 may extend helically around the stent 10 along the entire length of the stent 10. In some embodiments, when the stent 10 is in a fully relaxed state, the rungs 26 may extend substantially perpendicular to the longitudinal axis x-x of the stent 10, as shown in FIG. 6. In some embodiments, the rungs 26 may be between 0.1 mm and 10.0 mm in length in the relaxed, expanded configuration. In other examples, the rungs may have a length between 1 mm and 5 mm. In still other examples, the rungs 26 may have a length between 2 mm and 3 mm.

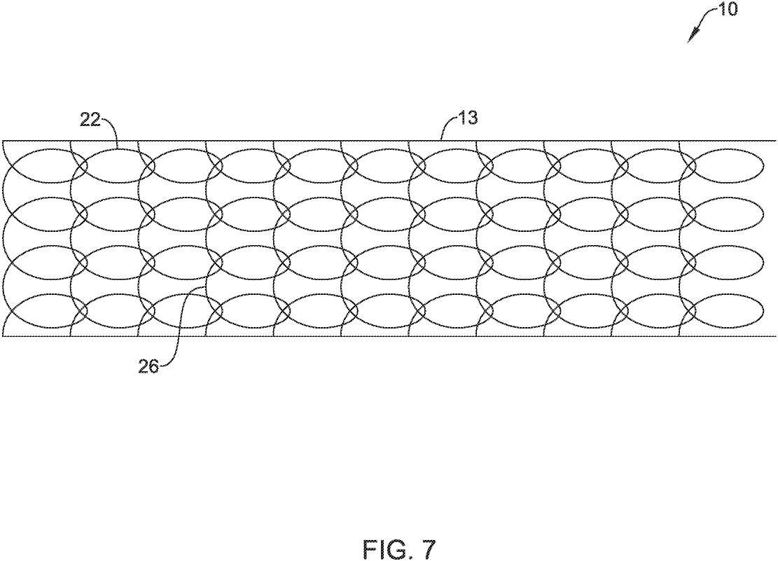

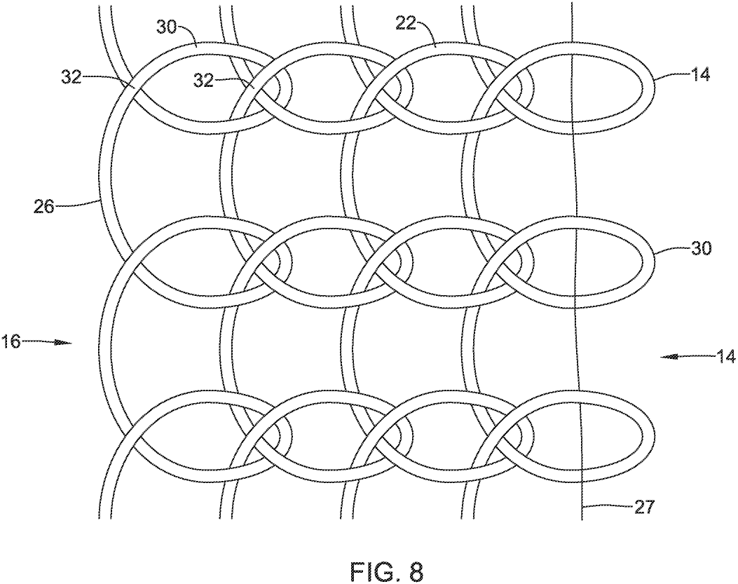

FIG. 7 illustrates the stent 10 in an elongated, compressed configuration disposed within a delivery sheath 13. When the stent 10 is collapsed and elongated as it is inserted into the delivery sheath 13, the helical interconnected twisted knit stitches 22 straighten into longitudinal columns, as shown in FIG. 7. The twisted knit stitches 22 elongate and the rungs 26 become shorter. The structure of the twisted knit stitches 22 in the elongated, compressed and constrained configuration is illustrated in FIG. 8. Each twisted knit stitch 22 may include a loop portion 30 and a crossed base region 32. The loop portions 30 may be wrapped around the crossed base regions 32 of longitudinally adjacent twisted knit stitches 22. The crossed base regions 32 are distal of the loop portions 30, such that at the distal end 16 of the stent, the crossed base regions 32 define an atraumatic structure, as shown in FIG. 8. While the loop portions 30 have an elongate or oval shape in the elongated, compressed configuration shown in FIG. 8, the loop portions 30 may have a generally circular shape in the relaxed, expanded configuration, as shown in FIG. 6. In some examples, the loops 130 may have a diameter of between 1 mm and 5 mm in the relaxed, expanded configuration. In other examples, the loops 130 may have a diameter of between 2 mm and 3 mm.

The proximal end 14 of the stent 10 may be defined by a series of free loop portions 30. In some embodiments, a tether or suture 27 may be threaded through the free loop portions 30 at the proximal end to facilitate removal of the stent 10. The retrieval suture 27 may be used to collapse and retrieve the stent 10, if so desired. For example, the retrieval suture 27 may be pulled like a drawstring to radially collapse the proximal end 14 of the stent 10 to facilitate removal of the stent 10 from a body lumen. The size of the free loop portions 30 at the proximal end may be increased or decreased to increase or decrease, respectively, the amount of tissue ingrowth at the proximal end achieved upon implantation of the stent 10.

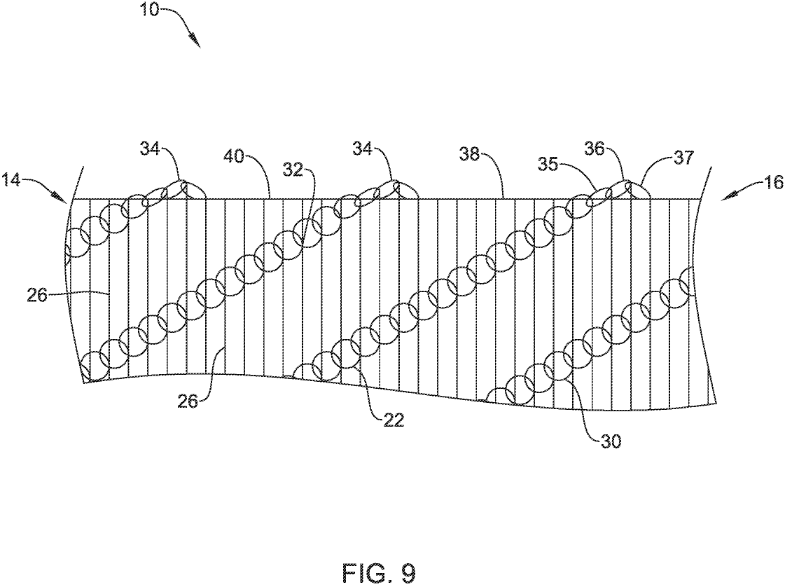

In the relaxed, expanded configuration, the rungs 26 define an outer surface 40 of the stent 10 and the crossed base regions 32 of the twisted knit stitches 22 extend radially outward from the outer surface 40, as shown in FIG. 9. The crossed base regions 32 form a raised ridge 34 extending helically around the stent 10. In some examples, the helical ridge 34 may have a longitudinal cross-sectional wave shape, with a proximal facing slope 35, a crest 36, and a pocket 37 facing a distal end 16 of the elongated tubular member. In some examples, the crest 36 may protrude from the outer surface 40 between 0.5 mm and 5.0 mm. In a particular example, the crest 36 may protrude 1.5 mm from the outer surface 40. The distance is essentially the diameter of the loop portion 30, and the minimum distance is dependent on the diameter of the filament 24. For example, 3 thou to 14 thou wires (0.0762 mm to 0.3556 mm) may be used as the filament 24. In one example, a 6 thou wire (0.1524 mm) was used as the filament 24.

The space between the helical ridges 34 may define channels 38 extending between crests 36 of adjacent ridges 34. The channels 38 may provide a drainage feature for the stent 10. The ridges 34 may engage the tissue wall, while leaving at least a portion of the channels 38 spaced from the tissue wall, providing for drainage of fluid along the entire length of the stent 10. A covering or graft disposed over the stent or within the lumen may aid in defining the channels 38.

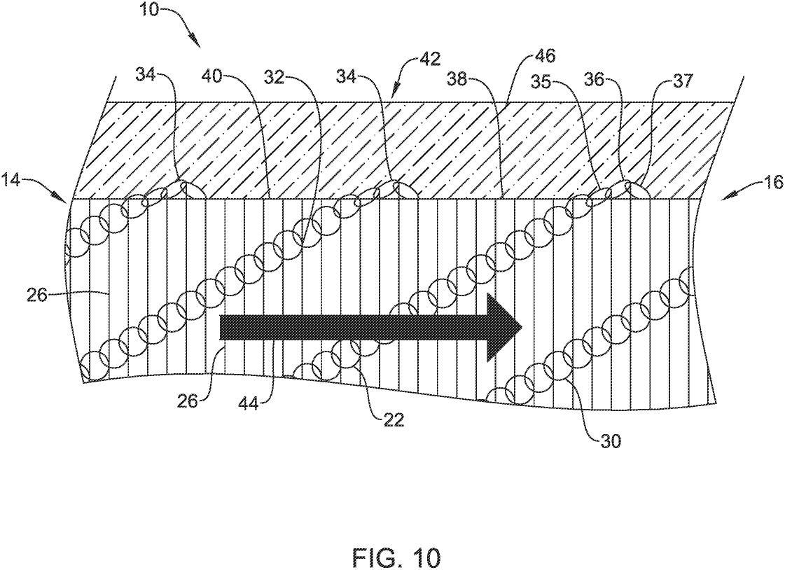

FIG. 10 illustrates a portion of the stent 10 disposed with an organ 42. The wave shape of the ridge 34 provides strong anti-migration properties in one direction and less in the opposite direction. The stent 10 may be loaded into a delivery sheath and placed in an organ of the body in the preferred orientation to optimize resistance to the migration force on the stent, as shown in FIG. 10. This unique anti-migration feature may also provide a benefit during removal of the stent, as during removal the stent may be pulled in the direction with less anti-migration properties. This feature may make removal of the stent easy for the physician without compromising any of the overall strong anti-migration properties of the stent.

When migration forces 44, such as peristalsis when the stent is disposed within the stomach or intestine, are exerted in a distal direction on the stent 10, the wave crest 36 provides resistance by pushing into the organ wall 46, and the pocket 37 engages a portion of the organ wall 46, as seen in FIG. 10, thereby preventing migration of the stent 10. The crest 36 is devoid of any sharp edge, barb, or quill. Rather, the crest 36 defines a smooth yet defined edge, as shown in FIG. 9. The anti-migration provided by the crest 36, is exhibited for each raised ridge 34 along the entire length of the stent 10. The wave shape of the ridge 34, in particular the gradual proximal facing slope 35, allows for removal of the stent 10 in the proximal direction without damage to the organ wall 46.

The twisted knit stitches 22, and in particular, the loop portions 30 may be configured to match the level of tissue ingrowth desired and/or required. For example, increased tissue ingrowth may be achieved by increasing the number of loop portions 30 around the circumference of the stent 10. The pitch and/or angle of the helices may also be increased, and the size of the loop portions 30 may be altered. The configuration of the loop portions 30 may have a more pronounced effect on the tissue ingrowth in stents having a bare metal composition, devoid of any covering or graft.

The peristaltic motion in the intestines occurs along the longitudinal surface of the organ wall. Existing parallel knitted stents have raised loops in a straight formation along the entire length of the stent. The forces transferred to such stents by peristalsis is thus constantly exerted on the entire length of the stent that is in contact with the intestinal wall. However, due to the helical ridges 34 of the stent 10, there is no direct transfer of force along the entire length of the stent that is in contact with the intestinal wall. Instead, the organ wall 46 exhibits force on the raised ridge 34 of the stent 10, but the force is intermittent, because no force is transferred to the outer surface 40 defined by the rungs 26 of the stent 10.

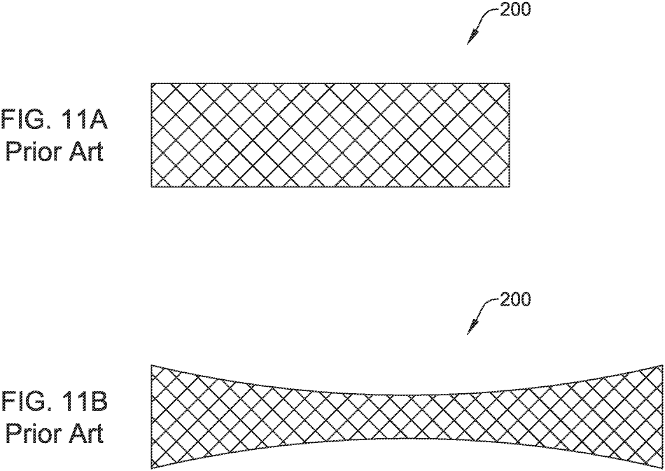

The configuration of the knit pattern as shown in FIG. 8, with a helical property may allow the stent to ‘store’ additional wire loops in a closed packed configuration that has a defined radial and axial flexibility. In comparison, a braid or a parallel knit (FIG. 4) has a design with a finite amount of material to operate and express their properties with. When braided or parallel knit stents are elongated the material has to reduce in diameter, radial or axial force to accommodate. FIGS. 11A and 11B demonstrate the performance of a traditional braided stent 200 as it moves between a relaxed, expanded configuration (FIG. 11A) to an elongated configuration (FIG. 11B). The braided stent 200 may accommodate a length change, for example from an initial length of 50 mm in FIG. 11A, the stent may be elongated by 30 mm, however this elongation results in a diameter that is reduced by 8 mm, as shown in the elongated state in FIG. 11B.

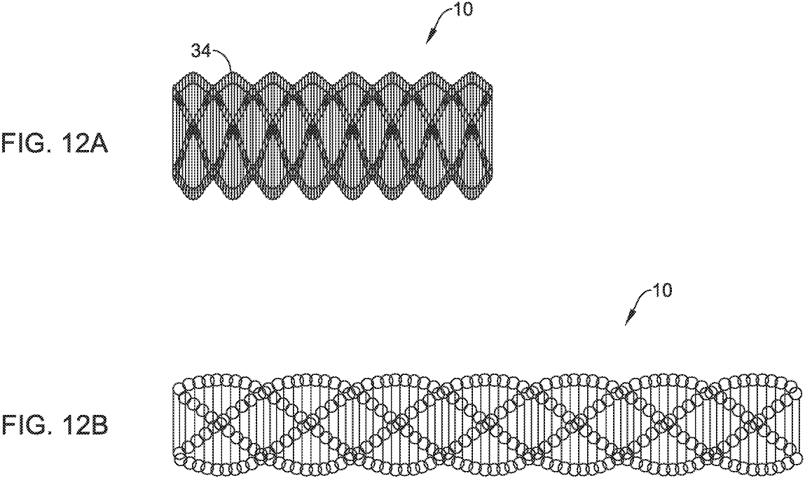

Elongation of the disclosed knitted pattern, as shown in FIG. 8, allows the stent to change in length without a significant reduction in diameter, and also to maintain radial force as the excess material ‘stored’ in the design is available, effectively over the span of the design as an ‘infinite’ braid. FIGS. 12A and 12B demonstrate the performance of the disclosed knitted pattern (FIG. 8) in the stent 10 as it moves between a relaxed, longitudinally contracted state (FIG. 12A) to an elongated state (FIG. 12B). The knitted stent 10 may accommodate a length change, for example from an initial length of 50 mm in FIG. 12A, the stent 10 may be elongated by 85 mm, with a change in diameter of 0 mm, as shown in FIG. 12B. The stent 10 may thus have a first longitudinal length and a first diameter in the relaxed, expanded configuration (FIG. 12A) and a second longitudinal length and a second diameter in the elongated configuration (FIG. 12B), where the first longitudinal length is less than the second longitudinal length, and the first and second diameters may be substantially the same. As a result, the stent 10 may be able to compensate for more movement between two organs, and may perform better in motile areas such as for the HGS procedure described above, while maintaining a substantially constant lumen diameter allowing for fluid flow through the stent. The stent 10 may behave like a spring in the deployed, relaxed and fully expanded configuration. This is not a property of conventional parallel knitted or braided metal stents.

As seen in FIG. 12A, in the relaxed configuration, the stent 10 has a series of helical ridges 34 of twisted knit stitches. When the stent 10 is partially elongated, the angle of the helical ridges 34 increases, as shown in FIG. 12B. As the stent 10 elongates, it may twist, and the angle of the helical ridges 34 may increase until the helical ridges 34 have straightened into longitudinal columns, as shown in FIG. 7. As the stent continues to elongate from the configuration in FIG. 12B to the configuration in FIG. 7, the outer diameter may be reduced. This may allow the stent 10 to be compressed into the delivery sheath 13.

During deployment, as the stent 10 is released from the delivery sheath 13, the stent 10 may twist in a corkscrew manner as it relaxes and moves from the delivery configuration in FIG. 7, through the elongated configuration of FIG. 12B to its original, relaxed helical shape of FIG. 12A. This corkscrew twisting motion during deployment may help the ends of the stent 10 engage the organ walls. This spring type expansion of the stent 10 means that the stent 10 will resist the peristaltic forces exerted on it, moving between the elongated configuration of FIG. 12B as the organs to which the ends of the stent are secured move relative one another during digestion, and returning to its original relaxed configuration and/or position of FIG. 12A after any elongation occurs during peristalsis and digestion. When the stent 10 experiences the peristaltic motion pushing along the stent 10, the part of the stent 10 expands radially ahead of the motion, similar to a spring. Once the peristaltic motion has passed down the length of the stent 10, the stent 10 will begin to return to its original position.

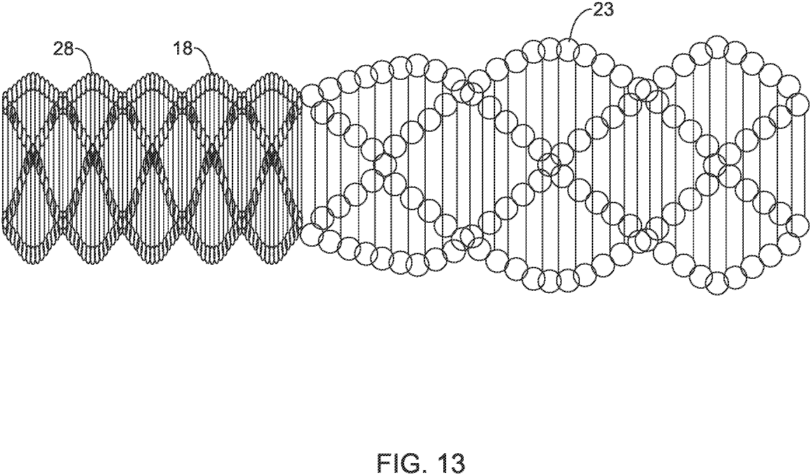

In some embodiments, the first end region 23 may include retention features or anti-migration flared regions having enlarged diameters relative to the intermediate region 18, as shown in FIG. 13. In other embodiments the second end region 28 is flared. Alternatively, both the first end region 23 and the second end region 28 may include flared regions. The flared end region may include a gradual increase in outer diameter, as shown in FIG. 13. In other embodiments, the change between the diameter in the intermediate region 18 and the diameter at the first and/or second end regions 23, 28, may be steeper. Anti-migration flared regions may be configured to engage an interior portion of the walls of the stomach, hepatic duct, or other body lumen. It is contemplated that a transition from the cross-sectional area of the intermediate region 18 to the retention features or flared regions may be gradual, sloped, or occur in an abrupt step-wise manner, as desired.

In some embodiments, the first anti-migration flared region may have a first outer diameter and the second anti-migration flared region may have a second outer diameter. In some instances, the first and second outer diameters may be approximately the same, while in other instances, the first and second outer diameters may be different. In some embodiments, the stent 10 may include only one or none of the anti-migration flared regions. For example, the first end region 23 may include an anti-migration flare while the second end region 28 may have an outer diameter similar to the intermediate region 18. It is further contemplated that the second end region 28 may include an anti-migration flare while the first end region 23 may have an outer diameter similar to an outer diameter of the intermediate region 18.

The stent 10 may be made in accordance with the methods described in U.S. Publication No. 2020/0214858 A1, the entirety of which is incorporated herein by reference.

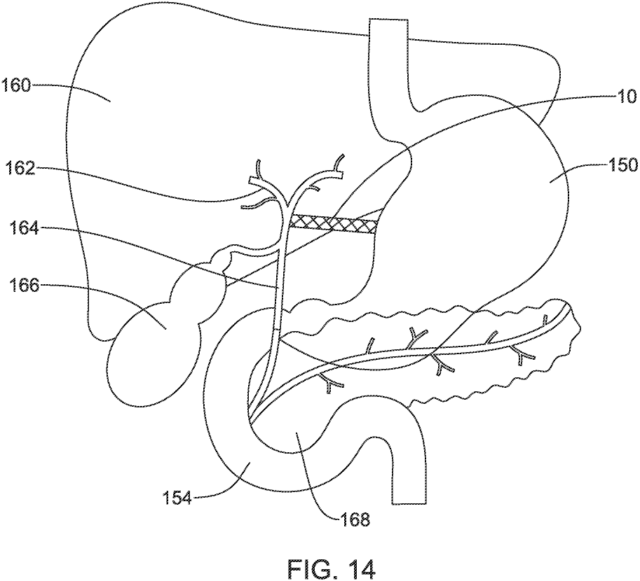

FIG. 14 illustrates the stent 10 as discussed above with a first end implanted in the stomach 150 and a second end implanted in the hepatic duct 162 for a hepaticogastrostomy (HGS) to drain bile into the stomach.

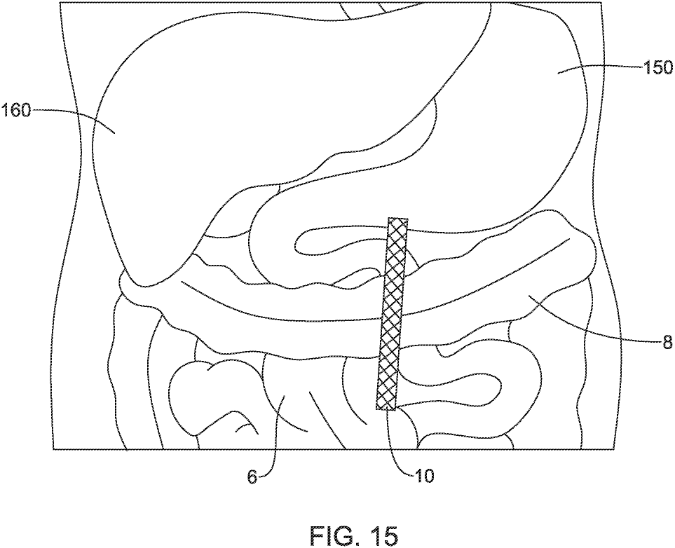

Similarly, the stent 10 may be used in a gastrojejeneum (GJ) bypass procedure. One GJ procedure involves inserting one end of the stent 10 into the stomach wall and the other end of the stent 10 into a distal portion of the small intestine 6, as shown in FIG. 15. The stent 10 creates an anastomosis between the small intestine 6 and the stomach 150, effectively rerouting the stomach contents directly into the small intestine, as shown in FIG. 15. As both the stomach wall and intestine experience peristaltic motion, this would be considered a highly motile application, which may benefit from the stent 10, where the bypass can be achieved while allowing an adaptable channel which retains its diameter throughout peristalsis.

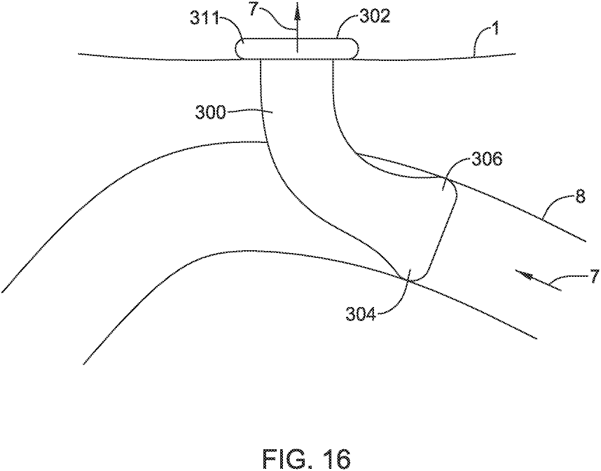

The stent described above may also be used when creating stomas within the intestinal tract. A stoma is an artificial opening in the large intestine or other region of the digestive tract, created during surgery, in order to bring the large intestine onto the surface of the abdomen. A loop stoma is formed when a loop of large intestine is brought through the abdominal wall and opened to reveal two ends. A stent 10 as described above may be used to connect the skin surface 1 with the large intestine 8. The use of a stent provides the advantage of leaving the large intestine 8 inside the body and requiring a smaller opening into the large intestine. Additionally, the stent 10 may eliminate the need to move a section of the large intestine 8 through the abdominal wall to the skin surface 1. In other embodiments, a stent 300 with the addition of one or more flange 311 may be used. As illustrated in FIG. 16, a flange 311 at the first end region 302 of the stent 300 may be disposed on the skin surface 1. In some embodiments, a second flange may be disposed on the inner surface of the skin or abdominal wall to further secure the stent 300 to the skin surface 1. The second end region 304 of the stent 300 may be inserted coaxially into the large intestine 8. In some embodiments, the second end region 304 of the stent 300 may include an outwardly flared region 306 to further secure the stent within the large intestine 8. The stent 300 may be able to accommodate any movements of the large intestine 8 during peristaltic motion. In some embodiments, the stent 300 may be covered with a fluid impermeable coating to direct the contents of the large intestine through the stent 300 in the direction indicated by arrows 7. In some embodiments an entirety of the stent 300 may be coated. This may be desired when the stoma is temporary and the stent 300 is to be removed after a period of time. In other embodiments, such as when the stoma is permanent, a portion of the second end region 304 may be uncovered (bare) to allow for tissue in-growth.

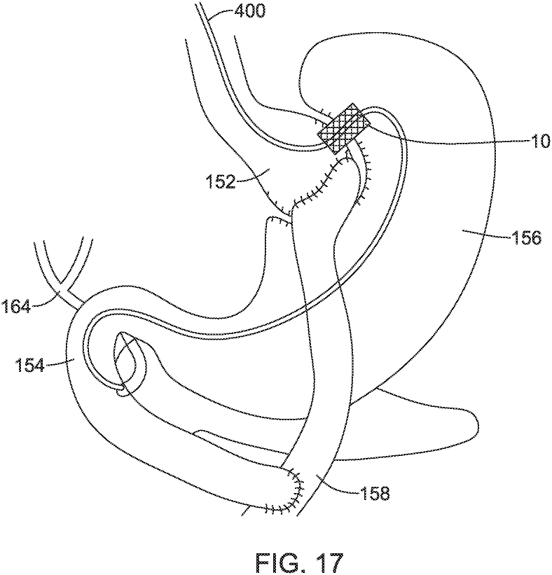

Additional applications for using the stent 10 as a bridging device between two spaced apart organs may be as part of an EDGE procedure. The internal EDGE (EUS-Directed trans Gastric ERCP) procedure is a new technique developed to perform ERCP in a completely endoscopic fashion in Roux-en-Y gastric bypass (RYGB) patients. In RYGB, the surgeon separates the upper portion of the stomach from the lower portion. The upper portion or pouch 152 is then connected to a limb 158 of the small intestine. See FIG. 17. The excluded stomach portion 156 and the duodenum 154 are effectively removed from the digestive process. In patients having undergone RYGB, a conventional ERCP is no longer possible because the bile duct 164 is no longer accessible through the stomach pouch 152.

Currently, the standard of care is to perform a combined surgical and endoscopic procedure to access the bile duct. The surgical part of this procedure may be avoided by using the stent 10 as described above to temporarily reverse a patient’s bypass to allow for to performance of a conventional ERCP. The stent 10 may be inserted between the pouch 152 and the excluded stomach portion 156 to temporarily provide access for an endoscope 400 to pass through the pouch 152 and the stent 10 into the excluded stomach portion 156 and the duodenum 154 to access the bile duct 164, as shown in FIG. 17. When the need for ERCP is complete, the stent 10 may be removed, and the bypass anatomy may be restored via endoscopic suturing. This entire procedure may all be performed completely from inside the body with an endoscope and can be performed on an outpatient basis. The proximity and relative motility of the RYGB pouch 152 and the excluded stomach portion 156 may be effectively bridged by the stent 10.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304 V, 304 L, and 316 LV stainless steel; mild steel; nickel-titanium alloy such as linear-elastic and/or superelastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005”). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention’s scope is, of course, defined in the language in which the appended claims are expressed.

What is claimed is:

1. A stent configured to connect two spaced apart anatomical locations, the stent comprising:
an elongated tubular member having a longitudinal axis, the elongated tubular member comprising at least one knitted filament forming a plurality of twisted knit stitches with rungs extending circumferentially between radially adjacent twisted knit stitches, wherein each twisted knit stitch is interconnected with a longitudinally adjacent twisted knit stitch forming a series of linked stitches, the elongated tubular member configured to move between a relaxed configuration and an elongated configuration;
wherein each of the plurality of twisted knit stitches is formed by a single filament defining a loop portion and a crossed base region; and
wherein the elongated tubular member has a first longitudinal length in the relaxed configuration and a second longitudinal length in the elongated configuration, wherein the first longitudinal length is less than the second longitudinal length.

2. The stent of claim 1, wherein the elongated tubular member has a first outer diameter in the relaxed configuration and a second outer diameter in the elongated configuration, wherein the first and second outer diameters are substantially the same.

3. The stent of claim 2, wherein the first and second outer diameters are defined by the rungs.

4. The stent of claim 1, wherein the loop portion of at least some of the twisted knit stitches is wrapped around the crossed base region of the longitudinally adjacent twisted knit stitch.

5. The stent of claim 1, wherein when in the relaxed configuration the rungs define an outer surface of the elongated tubular member and the crossed base region of each twisted knit stitch extends radially outward from the outer surface.

6. The stent of claim 5, wherein the crossed base regions form a raised ridge extending helically around the elongated tubular member in the relaxed configuration.

7. The stent of claim 1, wherein the at least one knitted filament is only a single knitted filament.

8. A method of providing an artificial bridge between a first organ and a second organ of a patient, comprising:
securing a first end of an expandable stent to the first organ and securing a second end of the stent to the second organ such that an interior of the first organ is in fluid communication with an interior of the second organ through the stent, wherein the first and second organs are spaced apart and at least one of the first and second organs moves relative to the other of the first and second organ during normal function of the first and second organs, wherein the stent comprises:
an elongated tubular member having a longitudinal axis, the elongated tubular member comprising at least one knitted filament forming a plurality of twisted knit stitches with rungs extending circumferentially between circumferentially adjacent twisted knit stitches, wherein each twisted knit stitch is interconnected with a longitudinally adjacent twisted knit stitch forming a series of linked stitches, the elongated tubular member configured to move between a relaxed configuration and an elongated configuration;
wherein each of the plurality of twisted knit stitches is formed by a single filament defining a loop portion and a crossed base region; and
wherein the elongated tubular member has a first longitudinal length in the relaxed configuration and a second longitudinal length in the elongated configuration, wherein the first longitudinal length is less than the second longitudinal length.

9. The method of claim 8, wherein the first organ is a hepatic duct and the second organ is the patient’s stomach.

10. The method of claim 9, wherein the stent incudes an uncoated portion and a coated portion, wherein the coated portion includes at least 70% of a length of the stent and the coated portion includes a fluid impervious coating, wherein the uncoated portion extends into the hepatic duct.

11. The method of claim 8, wherein the first organ is the patient’s stomach and the second organ is a portion of the patient’s small intestine.

12. The method of claim 8, wherein the first organ is the patient’s skin and the second organ is a portion of the patient’s large intestine.

13. The method of claim 12, wherein the first end of the stent includes a flange disposed on an outer surface of the skin.

14. The method of claim 13, wherein the second end of the stent is disposed coaxially within the large intestine.

15. The method of claim 14, wherein the second end of the stent disposed coaxially within the large intestine includes an outwardly flared region.

16. The method of claim 8, wherein the patient’s stomach includes a stomach pouch portion separated from an excluded stomach portion, and a Roux limb connecting the stomach pouch portion with the patient’s small intestine, wherein the first organ is the stomach pouch portion and the second organ is the excluded stomach portion.

17. The method of claim 16, wherein after the stent is secured, the method further comprises inserting an endoscope into the stomach pouch portion, through the stent into the excluded stomach portion, and into the patient’s duodenum, followed by performing an endoscopic retrograde cholangiopancreatography (ERCP).

18. The method of claim 17, wherein after ERCP is completed, the method further comprises removing the stent.

19. A method of draining fluids from a first organ to a second organ within a patient’s digestive tract, the method comprising:
inserting an implantable stent within the patient, wherein the implantable stent includes a tubular member formed from at least one knitted wire filament, forming a plurality of twisted knit stitches with rungs extending circumferentially between circumferentially adjacent twisted knit stitches, wherein each twisted knit stitch includes a loop portion and a crossed base region and is interconnected with a longitudinally adjacent twisted knit stitch forming a series of linked stitches, the tubular member configured to move between a relaxed configuration with a first longitudinal length and a first diameter, and an elongated configuration with a second longitudinal length and a second diameter, wherein the first longitudinal length is less than the second longitudinal length and the first and second diameters are substantially the same; and
positioning a first end of the tubular member within the first organ of the patient and a second end of the tubular member within the second organ of the patient, wherein the first and second organs are spaced apart from one another and move relative one another during digestion.

20. The method of claim 19, wherein when in the relaxed configuration the rungs define an outer surface of the tubular member and the crossed base region of each twisted knit stitch extends radially outward from the outer surface.
